SEQUENCE LISTING (69 sequences)

SEQ ID NO: 1 - protein, 19 aa, Artificial Sequence
  Synthetic
MEWSWVFLFFLSVTTGVMA

SEQ ID NO: 2 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 10 aa, Artificial Sequence
  Synthetic
DKTHTCPPCP

SEQ ID NO: 4 - protein, 365 aa, Artificial Sequence
  Synthetic
MEWSWVFLFFLSVTTGVMAAIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKGAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 115 aa, Homo sapiens
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 6 - protein, 115 aa, Artificial Sequence
  Synthetic
AAAALTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 7 - protein, 115 aa, Artificial Sequence
  Synthetic
AATAATEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 8 - protein, 115 aa, Artificial Sequence
  Synthetic
AATKAAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 9 - protein, 115 aa, Artificial Sequence
  Synthetic
AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHALFDRIRLVVT

SEQ ID NO: 10 - protein, 115 aa, Artificial Sequence
  Synthetic
AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLADRIRLVVT

SEQ ID NO: 11 - protein, 115 aa, Artificial Sequence
  Synthetic
AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFARIRLVVT

SEQ ID NO: 12 - protein, 115 aa, Artificial Sequence
  Synthetic
AATKLTEAKAELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 13 - protein, 115 aa, Artificial Sequence
  Synthetic
AATKLTEAKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFARIRLVVT

SEQ ID NO: 14 - protein, 115 aa, Artificial Sequence
  Synthetic
AATKLTEEAYALKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 15 - protein, 115 aa, Artificial Sequence
  Synthetic
AATKLTEEKYEAAEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 16 - protein, 115 aa, Artificial Sequence
  Synthetic
AATKLTEEKYELAAGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 17 - protein, 115 aa, Artificial Sequence
  Synthetic
AATKLTEEKYELAEGATLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 18 - protein, 115 aa, Artificial Sequence
  Synthetic
AATKLTEEKYELKAGQTLDVKCDYTLEKFASSQKAWQIIRDGEMPKTLACTERPSKNSHPVQVGRIILEDYHDHGLLRVRMVNLQAEDSGLYQCVIYQPPKEPHMLFDRIRLVVT

SEQ ID NO: 19 - protein, 115 aa, Artificial Sequence
  Synthetic
AATKLTEEKYELKEGQALDVKCDYTLEKFASSQKAWQIIRDGEMPKTLACTERPSKNSHPVQVGRIILEDYHDHGLLRVRMANLQVEDSGLYQCVIYQPPKEPHMLFDRIRLVVT

SEQ ID NO: 20 - protein, 115 aa, Artificial Sequence
  Synthetic
AATKLTEEKYELKEGQTLDVACDYTLEKFASSQKAWQIIRDGEMPKTLACTERPSKNSHPVQVGRIILEDYHDHGALRVRMVNLQVEDSGLYQCVIYQPPKEPHMLFDRIRLVVT

SEQ ID NO: 21 - protein, 115 aa, Artificial Sequence
  Synthetic
AATKLTEEKYELKEGQTLDVKCDYAAEKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 22 - protein, 115 aa, Artificial Sequence
  Synthetic
AATKLTEEKYELKEGQTLDVKCDYALAKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 23 - protein, 115 aa, Artificial Sequence
  Synthetic
AATKLTEEKYELKEGQTLDVKCDYALEAFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 24 - protein, 115 aa, Artificial Sequence
  Synthetic
AATKLTEEKYELKEGQTLDVKCDYTAAKFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 25 - protein, 115 aa, Artificial Sequence
  Synthetic
AATKLTEEKYELKEGQTLDVKCDYTLAAFASSQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 26 - protein, 115 aa, Artificial Sequence
  Synthetic
AATKLTEEKYELKEGQTLDVKCDYTLEKFAAAQKAWQIIRDGEMPKTLACTERPSKNSHPVQVGRIILEDYHDHGLLRVRMVNLQVEDSGLYQCVIYQPPKEPHMLFDRIRLVVT

SEQ ID NO: 27 - protein, 115 aa, Artificial Sequence
  Synthetic
AATKLTEEKYELKEGQTLDVKCDYTLEKFAASQKAWQIIRDGEMPKTLACTERASKNSHPVQVGRIILEDYHDHGLLRVRMVNLQVEDSGLYQCVIYQPPKEPHMLFDRIRLVVT

SEQ ID NO: 28 - protein, 115 aa, Artificial Sequence
  Synthetic
AATKLTEEKYELKEGQTLDVKCDYTLEKFASSQKAWQAARDGEMPKTLACTERPSKNSHPVQVGRIILEDYHDHGLLRVRMVNLQVEDSGLYQCVIYQPPKEPHMLFDRIRLVVT

SEQ ID NO: 29 - protein, 115 aa, Artificial Sequence
  Synthetic
AATKLTEEKYELKEGQTLDVKCDYTLEKFASSQKAWQAIADGEMPKTLACTERPSKNSHPVQVGRIILEDYHDHGLLRVRMVNLQVEDSGLYQCVIYQPPKEPHMLFDRIRLVVT

SEQ ID NO: 30 - protein, 115 aa, Artificial Sequence
  Synthetic
AATKLTEEKYELKEGQTLDVKCDYTLEKFASSQKAWQAIRDGEMPATLACTERPSKNSHPVQVGRIILEDYHDHGLLRVRMVNLQVEDSGLYQCVIYQPPKEPHMLFDRIRLVVT

SEQ ID NO: 31 - protein, 115 aa, Artificial Sequence
  Synthetic
AATKLTEEKYELKEGQTLDVKCDYTLEKFASSQKAWQAIRDGEMPKTAACTERPSKNSHPVQVGRIILEDYHDHGLLRVRMVNLQVEDSGLYQCVIYQPPKEPHMLFDRIRLVVT

SEQ ID NO: 32 - protein, 115 aa, Artificial Sequence
  Synthetic
AATKLTEEKYELKEGQTLDVKCDYTLEKFASSQKAWQIARDGEMAKTLACTERPSKNSHPVQVGRIILEDYHDHGLLRVRMVNLQVEDSGLYQCVIYQPPKEPHMLFDRIRLVVT

SEQ ID NO: 33 - protein, 115 aa, Artificial Sequence
  Synthetic
AATKLTEEKYELKEGQTLDVKCDYTLEKFASSQKAWQIARDGEMPKTLACTERPSKNSHPVQVGRIILEDYHDHGLLRVRMVNLQVEDSGAYQCVIYQPPKEPHMLFDRIRLVVT

SEQ ID NO: 34 - protein, 115 aa, Artificial Sequence
  Synthetic
AATKLTEEKYELKEGQTLDVKCDYTLEKFASSQKAWQIIRDGAAPKTLACTERPSKNSHPVQVGRIILEDYHDHGLLRVRMVNLQVEDSGLYQCVIYQPPKEPHMLFDRIRLVVT

SEQ ID NO: 35 - protein, 115 aa, Artificial Sequence
  Synthetic
AATKLTEEKYELKEGQTLDVKCDYTLEKFASSQKAWQIIRDGAMPATLACTERPSKNSHPVQVGRIILEDYHDHGLLRVRMVNLQVEDSGLYQCVIYQPPKEPHMLFDRIRLVVT

SEQ ID NO: 36 - protein, 115 aa, Artificial Sequence
  Synthetic
AATKLTEEKYELKEGQTLDVKCDYTLEKFASSQKAWQIIRDGEAAKTLACTERPSKNSHPVQVGRIILEDYHDHGLLRVRMVNLQVEDSGLYQCVIYQPPKEPHMLFDRIRLVVT

SEQ ID NO: 37 - protein, 115 aa, Artificial Sequence
  Synthetic
AATKLTEEKYELKEGQTLDVKCDYTLEKFASSQKAWQIIRDGEAPKTLACTERPSKNSHPVQVGRIILEDYHDHGLLRVRMVNLQVEDSGLYQCVIYQPPKAPHMLFDRIRLVVT

SEQ ID NO: 38 - protein, 115 aa, Artificial Sequence
  Synthetic
AATKLTEEKYELKEGQTLDVKCDYTLEKFASSQKAWQIIRDGEMAATLACTERPSKNSHPVQVGRIILEDYHDHGLLRVRMVNLQVEDSGLYQCVIYQPPKEPHMLFDRIRLVVT

SEQ ID NO: 39 - protein, 115 aa, Artificial Sequence
  Synthetic
AATKLTEEKYELKEGQTLDVKCDYTLEKFASSQKAWQIIRDGEMPATAACTERPSKNSHPVQVGRIILEDYHDHGLLRVRMVNLQVEDSGLYQCVIYQPPKEPHMLFDRIRLVVT

SEQ ID NO: 40 - protein, 115 aa, Artificial Sequence
  Synthetic
AATKLTEEKYELKEGQTLDVKCDYTLEKFASSQKAWQIIRDGEMPKTAACTERPSKNSHPVQVGRAILEDYHDHGLLRVRMVNLQVEDSGLYQCVIYQPPKEPHMLFDRIRLVVT

SEQ ID NO: 41 - protein, 115 aa, Artificial Sequence
  Synthetic
AATKLTEEKYELKEGQTLDVKCDYTLEKFASSQKAWQIIRDGEMPKTLACTARPSANSHPVQVGRIILEDYHDHGLLRVRMVNLQVEDSGLYQCVIYQPPKEPHMLFDRIRLVVT

SEQ ID NO: 42 - protein, 115 aa, Artificial Sequence
  Synthetic
AATKLTEEKYELKEGQTLDVKCDYTLEKFASSQKAWQIIRDGEMPKTLACTERPSKAAHPVQVGRIILEDYHDHGLLRVRMVNLQVEDSGLYQCVIYQPPKEPHMLFDRIRLVVT

SEQ ID NO: 43 - protein, 115 aa, Artificial Sequence
  Synthetic
AATKLTEEKYELKEGQTLDVKCDYTLEKFASSQKAWQIIRDGEMPKTLACTERPSKNSAAVQVGRIILEDYHDHGLLRVRMVNLQVEDSGLYQCVIYQPPKEPHMLFDRIRLVVT

SEQ ID NO: 44 - protein, 115 aa, Artificial Sequence
  Synthetic
AATKLTEEKYELKEGQTLDVKCDYTLEKFASSQKAWQIIRDGEMPKTLACTERPSKNSHAAQVGRIILEDYHDHGLLRVRMVNLQVEDSGLYQCVIYQPPKEPHMLFDRIRLVVT

SEQ ID NO: 45 - protein, 115 aa, Artificial Sequence
  Synthetic
AATKLTEEKYELKEGQTLDVKCDYTLEKFASSQKAWQIIRDGEMPKTLACTERPSKNSHAVAVGRIILEDYHDHGLLRVRMVNLQVEDSGLYQCVIYQPPKEPHMLFDRIRLVVT

SEQ ID NO: 46 - protein, 115 aa, Artificial Sequence
  Synthetic
AATKLTEEKYELKEGQTLDVKCDYTLEKFASSQKAWQIIRDGEMPKTLACTERPSKNSHAVQVGRIALEDYHDHGLLRVRMVNLQVEDSGLYQCVIYQPPKEPHMLFDRIRLVVT

SEQ ID NO: 47 - protein, 115 aa, Artificial Sequence
  Synthetic
AATKLTEEKYELKEGQTLDVKCDYTLEKFASSQKAWQIIRDGEMPKTLACTERPSKNSHPAAVGRIILEDYHDHGLLRVRMVNLQVEDSGLYQCVIYQPPKEPHMLFDRIRLVVT

SEQ ID NO: 48 - protein, 115 aa, Artificial Sequence
  Synthetic
AATKLTEEKYELKEGQTLDVKCDYTLEKFASSQKAWQIIRDGEMPKTLACTERPSKNSHPAQAGRIILEDYHDHGLLRVRMVNLQVEDSGLYQCVIYQPPKEPHMLFDRIRLVVT

SEQ ID NO: 49 - protein, 115 aa, Artificial Sequence
  Synthetic
AATKLTEEKYELKEGQTLDVKCDYTLEKFASSQKAWQIIRDGEMPKTLACTERPSKNSHPVAAGRIILEDYHDHGLLRVRMVNLQVEDSGLYQCVIYQPPKEPHMLFDRIRLVVT

SEQ ID NO: 50 - protein, 115 aa, Artificial Sequence
  Synthetic
AATKLTEEKYELKEGQTLDVKCDYTLEKFASSQKAWQIIRDGEMPKTLACTERPSKNSHPVAVGRIALEDYHDHGLLRVRMVNLQVEDSGLYQCVIYQPPKEPHMLFDRIRLVVT

SEQ ID NO: 51 - protein, 115 aa, Artificial Sequence
  Synthetic
AATKLTEEKYELKEGQTLDVKCDYTLEKFASSQKAWQIIRDGEMPKTLACTERPSKNSHPVQAGRAILEDYHDHGLLRVRMVNLQVEDSGLYQCVIYQPPKEPHMLFDRIRLVVT

SEQ ID NO: 52 - protein, 115 aa, Artificial Sequence
  Synthetic
AATKLTEEKYELKEGQTLDVKCDYTLEKFASSQKAWQIIRDGEMPKTLACTERPSKNSHPVQVGRAALEDYHDHGLLRVRMVNLQVEDSGLYQCVIYQPPKEPHMLFDRIRLVVT

SEQ ID NO: 53 - protein, 115 aa, Artificial Sequence
  Synthetic
AATKLTEEKYELKEGQTLDVKCDYTLEKFASSQKAWQIIRDGEMPKTLACTERPSKNSHPVQVGRIALEDYHDHGLLRVRMANLQVEDSGLYQCVIYQPPKEPHMLFDRIRLVVT

SEQ ID NO: 54 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHAAGLLRVRMVNLQVEDSGLYQCVIYQPPKEPHMLFDRIRLVVT

SEQ ID NO: 55 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDAGALRVRMVNLQVEDSGLYQCVIYQPPKEPHMLFDRIRLVVT

SEQ ID NO: 56 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMAALQVEDSGLYQCVIYQPPKEPHMLFDRIRLVVT

SEQ ID NO: 57 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQAADSGLYQCVIYQPPKEPHMLFDRIRLVVT

SEQ ID NO: 58 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQAAKEPHMLFDRIRLVVT

SEQ ID NO: 59 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPAAEPHMLFDRIRLVVT

SEQ ID NO: 60 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPAAPHMLFDRIRLVVT

SEQ ID NO: 61 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKAAHMLFDRIRLVVT

SEQ ID NO: 62 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEAAMLFDRIRLVVT

SEQ ID NO: 63 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPAALFDRIRLVVT

SEQ ID NO: 64 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHALADRIRLVVT

SEQ ID NO: 65 - protein, 115 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLAARIRLVVT

SEQ ID NO: 66 - protein, 117 aa, Artificial Sequence
  Synthetic
AATKLTEEAYALKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLAVTKG

SEQ ID NO: 67 - protein, 117 aa, Artificial Sequence
  Synthetic
AATKLTEEKYELKEGQTLDVKCDYTLEKFASSQKAWQAIAAGEMPKTLACTERPSKNSHPVQVGRIILEDYHDHGLLRVRMVNLQVEDSGLYQCVIYQPPKEPHMLFDRIRLVVTKG

SEQ ID NO: 68 - protein, 117 aa, Artificial Sequence
  Synthetic
AATKLTEEKYELKEGQTLDVKCDYTLEKFASSQKAWQIIRAGEMPKTLACTERPSKNSHPVQVGRIILEDYHDHGLLRVRMVNLQVEDSGLYQCVIYQPPKEPHMLFDRIRLVVTKG

SEQ ID NO: 69 - protein, 117 aa, Artificial Sequence
  Synthetic
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLAVTKG

(19) United States
(12) Patent Application Publication
(10) Pub. No.: US 2023/0176071 A1
(43) Pub. Date: Jun. 8, 2023

(54) ARRAYS AND METHODS FOR IDENTIFYING BINDING SITES ON A PROTEIN

(71) Applicant: UCB BIOPHARMA SRL, Brussels (BE)

(72) Inventors: Anastasios SPILIOTOPOULOS, Slough (GB); David James MCMILLAN, Slough (GB); Michael John Wright, Slough (GB); Sebastian KELM, Slough (GB); Xiaofeng LIU, Slough (GB); Daniel John LIGHTWOOD, Slough (GB)

(21) Appl. No.: 17/923,904

(22) PCT Filed: May 6, 2021
(86) PCT No.: PCT/EP2021/061949

§ 371 Date: Nov. 7, 2022

(30) Foreign Application Priority Data
May 8, 2020 (EP) 20173658.4

(51) Int. Cl. G01N 33/68

(52) U.S. Cl. CPC G01N 33/6857

(57) ABSTRACT

A method of identifying of amino-acid residues on a target protein that form a binding site of a molecule of interest. Such method relies on selection of relevant patches of solvent-accessible residues and testing of an array of mutated proteins for changes of binding properties. Such method is useful for determining binding sites (epitopes) of antibodies, ligands and related molecules.

The present disclosure relates to methods for identifying binding sites on a protein. In particular such methods can be used with any molecule of interest, and are very useful when, for example, determining epitopes of antibody molecules.

### BACKGROUND

A binding site of a ligand or an epitope of an antibody can be identified using a variety of methods. Examples of such methods include screening peptides of varying lengths derived from full length target protein for binding to the antibody or a ligand and identify the smallest fragment that can specifically bind to an antibody containing the sequence of the epitope recognized by the antibody. However, such methods suffer from being imprecise and only provide an indication of a region of the target protein. Peptides that bind such antibody can be identified by using, for example, mass spectrometric analysis.

NMR spectroscopy or X-ray crystallography can be used to identify the binding site bound by a molecule. Typically, when the binding site determination is performed by X-ray crystallography, amino acid residues of the antigen within 4-5 Å from the amino acid of the molecule are considered to be amino acid residues part of the binding site.

One of the methods that can be used for identification of residues or regions of a binding site of a target protein is alanine scanning mutagenesis (Cunningham and Wells (1989) Science, 244: 1081-1085). In this method, a residue or a number of target residues are identified and replaced by alanine to determine whether the interaction of the antibody with antigen is affected. But using single Ala mutation poses some issues such as the lack of a measurable effect on the kinetics of an antibody.

The present disclosure addresses the issues posed with the use of single Ala mutants and provides a method that uses multiple Ala substitutions that improves the sensitivity and allows for better detection of residues involved in binding and providing higher sensitivity by increasing the effect on the kinetics of binding of a molecule of interest to the target protein (either association or dissociation constants).

### SUMMARY

The present disclosure provides a method of identifying amino-acid residues on a target protein that form a binding site of a molecule of interest, said method comprising:
a) obtaining 3D structure information for the target protein;
b) identifying, using obtained 3D structural data, the amino-acid residues which are within the accessible surface area;
c) for each of the identified amino-acids selecting 1 or 2 amino-acids which are within a predetermined distance from the identified amino-acid and are within the accessible surface area, whereby such combination of amino-acid residues forms a patch of 2 or 3 amino acids (patch);
d) selecting, from the large number of generated possible patches, a set of representative patches that cover the majority of the target protein's accessible surface area, while minimizing the number of patches likely to cause the target protein to misfold by eliminating patches that result in
   i. the breakage of 3 or more hydrogen bonds in the target protein;
   ii. the breakage of 2 or more salt bridges in the target protein; and
   iii. the exposure of hydrophobic surface of the target protein above a predetermined threshold;
e) producing a set of mutant proteins, wherein each of the mutant proteins comprises a mutated sequence of the target protein, wherein each of the mutated sequences comprises a single mutated patch of amino acids identified in step (d), and wherein each of the amino acids of the patch is substituted by another amino-acid;
f) measuring binding properties of each of the mutant proteins; and
g) identifying the patches that demonstrate decreased binding properties of the molecule of interest to the corresponding mutant protein comprising such a patch, wherein the residues in such patches are identified as being a part of a binding site of the molecule of interest.

The present disclosure also provides an array of proteins, wherein such array comprises a multiplicity of mutant protein sequences of a target protein, each such protein comprising a patch of 2 or 3 amino-acid substitutions for another amino-acid (patch) compared to the parent sequence of the target protein, and wherein such substitutions have been introduced into the residues of the accessible surface area, and wherein the residues in each patch are within a predetermined spatial distance from each other, and wherein the patches do not result in
i. the breakage of 3 or more hydrogen bonds in the target protein;
ii. the breakage of 2 or more salt bridges in the target protein; and
iii. the exposure of hydrophobic surface of the target protein above a predetermined threshold;

### BRIEF DESCRIPTION OF THE DRAWINGS

The present disclosure is described below by reference to the following drawings, in which:

FIG. 1 is a schematics representation of the method utilizing (A) 96 well plate where each well contains an alanine mutant protein clone fused to human Fc. Well A1 is used for the wt protein (control). In each well an octet tip coated with antibody is dipped to capture the protein and hence “load” the sensor; (B) the sensor tip showing the surface coated with anti-human Fc IgG; (C) BLI machine where the plate is ultimately loaded and kinetic parameters are analysed

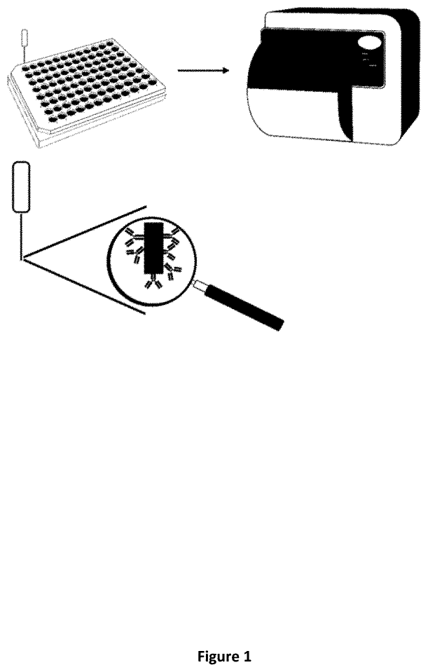

FIG. 2 is a raw data plot showing the capturing level (Y axis units in nm) of different mutant protein clones on a sensor over time (x axis units in seconds).

FIG. 3 is a raw data plot showing an exemplary response level and the kinetics profile upon binding of an antibody of interested to the wild-type (wt) protein and six alanine mutant clones (numbered 1 to 6). The effect of the mutations on antibody binding is exemplified as either a lack of response (clones 4, 5, and 6) or fast dissociation rate (clones 1, 2 and 3).

### DETAILED DESCRIPTION

### Abbreviations

The following abbreviations are used herein: mAb, monoclonal antibody; IgG, immunoglobulin G; Fab, fragment antigen binding; Fc, fragment crystallizeable; Fv, fragment variable; VL, variable domain of a light chain; VH, variable domain of a heavy chain; CHL first domain in constant portion of a heavy chain; CH2, second domain in constant portion of a heavy chain; CH3, third domain in constant portion of a heavy chain.

TABLE 1
Amino acids abbreviations

| Abbreviation | 1 letter abbreviation | Amine acid name |
|---|---|---|
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Len | L | Leucine |
| Lys | K | Lysine |
| Met | M | Methionine |
| Phe | F | Phenylalanine |
| Pro | P | Proline |
| Pyl | O | Pyrrolysine |
| Ser | S | Serine |
| Sec | U | Selenocysteine |
| Thr | T | Threonine |
| Trp | W | Tryptophan |
| Tyr | Y | Tyrosine |
| Val | V | Valine |

### Definitions

The following definitions are used throughout the description.

The term “amino acid” as used herein refers to one of the 20 naturally occurring amino acids that are coded for by DNA and RNA.

The term “pKa” is defined as the negative logarithm of the ionization constant Kₐ of an acid (pKₐ=−10 log Kₐ). The determination of the ionization constant Ka and its definition is explained in “Physical Chemistry”, F. Daniels and R. Alberty, Second Edition, 1961, John Wiley and Sons, Inc., pages 364, 365, 428-430.

The term “Kd” as used herein refers to the constant of dissociation which is obtained from the ratio of Kd to Ka (i.e. Kd/Ka) and is expressed as a molar concentration (M). Kd and Ka refers to the dissociation rate and association rate, respectively, of a particular molecule of interest—target protein interaction. Kd values can be determined using methods well established in the art.

The term “salt bridge” used herein refers to a link between electrically charged acidic and basic groups, especially on different parts of a large molecule such as a protein. The salt bridge most often arises from the anionic carboxylate (RCOO⁻) of either aspartic acid or glutamic amino acid and the cationic ammonium (RNH³⁺) from lysine or the guanidinium (RNHC(NH₂)²⁺) of arginine Although these are the most common, other residues with ionizable side chains such as histidine, tyrosine, and serine can also participate in salt bridge formation, depending on outside factors perturbing their pKa's.

The term “accessible surface area” (ASA) or “solvent-accessible surface area” is the surface area of a biomolecule that is accessible to a solvent.

The term “target protein” refers to a protein to which a particular molecule of interest, such as an antibody, binds. In the context of the present disclosure the term refers to target proteins that are modified in order to establish the residues of such proteins that are involved in binding of a molecule of interest.

The term “protein” herein is meant at least two covalently attached amino acids, which includes proteins, polypeptides, oligopeptides and peptides. The term protein is intended to include sequences of amino acids whose chain length is sufficient to produce higher levels of secondary and/or tertiary and/or quaternary structure The term protein also includes multi-domain proteins and proteins that comprise more than one amino-acid sequence (chain), such as multimeric proteins. The peptidyl group may comprise naturally occurring amino acids and peptide bonds, or synthetic peptidomimetic structures, i.e. “analogs”, such as peptoids (see Simon et al., PNAS USA 89(20):9367 (1992)). The amino acids may either be naturally occurring or synthetic. Proteins may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

The term “protein domain” or “domain” refers to any identifiable longer contiguous subsequence of a protein that can fold, function and exist independently of the rest of the protein chain or structure. A domain is characterized by a three-dimensional structure and can be often stable and folded independently of other domains.

The term “array” as used herein refers to a collection of samples comprising mutant proteins and, optionally, controls. Preferably each sample represents a spatially separated addressable element. Such elements can be spatially addressable, such as arrays contained within microtiter plates, or immobilized on planar surfaces where each element is present at distinct X and Y coordinates, or represent of a collection of tubes or other containers each comprising individual mutant protein. For spatial addressability, also known as coding, the position of the element is fixed, and that position is correlated with the identity, thereby allowing identification of the specificity of the mutant proteins contained within the sample to be tested in such array. Typically an array has at least 3 or more samples.

The term “molecule of interest” refers to a molecule for which the binding to the Target protein is being accessed. Typically such molecule of interest would be a protein or an antibody that binds to the target protein.

The term “protein binding site” or “binding site” as used herein refers to the part of a target protein where the molecule of interest binds. The binding partner (the molecule of interest) could be a ligand or a receptor of such target protein.

The term “epitope” is used interchangeably for both conformational and linear epitopes, where a conformational epitope is composed of discontinued sections of the antigen's amino acid primary sequence and a linear epitope is formed by a sequence formed by continuous amino acids. Epitopes are generally refer to binding of an antibody to its target antigen (protein of interest).

The term “ligand” as used herein refers to any ligand that will bind to or be bound by the target protein. The ligand may be an amino acid molecule, a polypeptide, a peptide or a chemical derivative thereof, or a combination thereof. The ligand may be a polynucleotide molecule. The ligand may be an antibody.

The term “antibody” herein refers to multi-domain antibodies. The term “antibody” includes traditional antibodies as well as antibody derivatives and fragments. In general, the term “antibody” includes any polypeptide that includes at least one constant domain, including, but not limited to, CH1, CH2, CH3 and CH4. Traditional antibody structural units typically comprise a tetramer. Each tetramer is typically composed of two identical pairs of polypeptide chains, each pair having one “light” (L) and one “heavy” (H) chain. Human light chains are classified as kappa and lambda light chains. Each heavy chain is comprised of a heavy chain variable region (abbreviated herein as HCVR or VH) and a heavy chain constant region. The heavy chain constant region of an IgG subclass of immunoglobulins, for example, is comprised of three domains, CHI, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding fragment” refers to functionally active fragments of antibodies and are molecules that contain an antigen binding domain that specifically binds an antigen. Features described herein with respect to antibodies also apply to antibody fragments unless context dictates otherwise. Antigen-binding fragments of antibodies include single chain antibodies (e.g. scFv, and dsscfv), Fab, Fab′, F(ab′)2, Fv, single domain antibodies or nanobodies (e.g. VH or VL, or VHH or VNAR). Other antibody fragments for use in the present disclosure include the Fab and Fab′ fragments described in International patent applications WO2011/117648, WO2005/003169, WO2005/003170 and WO2005/003171. An alternative antigen-binding fragment comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin) Such antibody fragments are described in International Patent Application Publication No, WO2015/197772.

The term “monoclonal antibody” as used herein refers to an antibody obtained from a population of substantially homogeneous antibodies. Thus, the modifier “monoclonal” indicates the character of the antibody as not being a mixture of different antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences.

The term “Fc”, “Fc fragment”, and “Fc region” are used interchangeably to refer to the C-terminal region of an antibody comprising the constant region of an antibody excluding the first constant region immunoglobulin domain. Thus, Fc refers to the last two constant domains, CH2 and CH3, of IgA, IgD, and IgG, or the last three constant domains of IgE and IgM, and the flexible hinge N-terminal to these domains. The human IgG1 heavy chain Fc region is defined herein to comprise residues C226 to its carboxyl-terminus, wherein the numbering is according to the EU index as in Kabat. In the context of human IgG1, the lower hinge refers to positions 226-236, the CH2 domain refers to positions 237-340 and the CH3 domain refers to positions 341-447 according to the EU index as in Kabat. The corresponding Fc region of other immunoglobulins can be identified by sequence alignments.

The term “human Fc region” or “human Fc domain” refer to Fc region which possesses an amino acid sequence which corresponds to that of an antibody produced by a human or a human cell or derived from a non-human source that utilizes human antibody repertoires or other human antibody-encoding sequences.

The term “modification” used herein refers to any amino acid substitution, insertion, and/or deletion in a polypeptide sequence or to a chemical alteration of an amino acid. The term “amino acid modification” herein means an amino acid substitution, insertion, and/or deletion in a polypeptide sequence. For clarity, unless otherwise noted, the amino acid is any amino acid coded for by DNA, e.g. the 20 amino acids that have codons in DNA and RNA.

The term “amino acid substitution” or “substitution” or “amino acid replacement” herein means replacement of an amino acid at a particular position in a parent polypeptide sequence with a different amino acid. In particular, in some embodiments, the substitution is to an amino acid that is not naturally occurring at the particular position, either not naturally occurring within the organism or in any organism.

The term “mutant protein(s)” as used herein means a protein that differs from the reference protein (also referred to a wild-type protein) by at least one amino acid modification. The term protein variant may refer to the protein itself, a composition comprising the protein, or the amino sequence that encodes it. Preferably, the protein variant has at least two amino acid modifications compared to the parent protein.

By “position” as used herein is meant a location in the sequence of a protein. Positions may be numbered sequentially, or according to an established format, for example the EU index for antibody numbering.

### Identification of Binding Sites

The methods that rely on mutating a single residue on the protein surface (1 Ala array) minimize the chances of having a misfolded mutant protein and can identify key epitope residues. However, single alanine substitution in many cases is not sufficient to give a clear effect on ligand's binding kinetics (a subtle effect on the dissociation constant may or may not be observed). Therefore, by mutating more than one surface residue, for example in 2 and 3 alanine arrays, this problem is addressed by creating an alanine patch. The caveat of this approach is that the percentage of misfolded clones might increase, but these can easily be excluded from such array to avoid false positives.

The present disclosure for the first time demonstrates that using multiple substitutions of amino acids located in close proximity on the solvent-accessible surface area of the protein by another amino-acid (such as Ala) allows to determine more precisely the residues that are important for binding of a molecule of interest or otherwise form a part of an epitope for such molecule of interest. In particular such molecule of interest is a protein or a peptide, such as, for example, a ligand or a receptor of the target protein. More specifically such method is suitable for determination of an epitope of an antibody molecule or any antigen-binding fragment of such antibody.

Alanine is useful as a substitute amino acid due its small side chain (CH3). Alternatively, glycine can also be used, however the side chain consists of only a H atom and is therefore extremely flexible. In principle any amino acid with a small side chain can be used.

The present disclosure provides a method of identifying amino-acid residues on a target protein that form a binding site of a molecule of interest, said method comprising:
a) obtaining 3D structure information for the target protein;
b) identifying, using obtained 3D structural data, the amino-acid residues which are within the accessible surface area;
c) for each of the identified amino-acids selecting 1 or more amino-acids which are within a predetermined distance from the identified amino-acid and are within the accessible surface area, whereby such combination of amino-acid residues forms a patch of 2 or more amino acids (patch);
d) selecting, from the large number of possible patches, a set of representative patches that cover the majority of the target protein's accessible surface area, while minimizing the number of patches likely to cause the target protein to misfold;
e) producing a set of mutant proteins, wherein each of the mutant proteins comprises a mutated sequence of the target protein, wherein each of the mutated sequences comprises a single mutated patch of amino acids identified in step (d), and wherein each of the amino acids of the patch is substituted by another amino-acid;
f) measuring binding properties of each of the mutant proteins; and
g) identifying the patches that demonstrate decreased binding properties of the molecule of interest to corresponding mutant protein comprising such patch, wherein the residues in such patches are identified as being a part of a binding site of the molecule of interest.

The method and its various embodiments are described further herein.

### Selecting Residues for Substitutions

In order to identify the amino-acid residues for producing mutant versions of the protein of interest, 3D structure data needs to be obtained for such a protein of interest. Such data might already be available in the form of a PDB structure (electronic file containing structural data) of the relevant protein of interest or its relevant domain. Alternatively, such structural data can be obtained using the techniques known to the skilled person. Such techniques include X-ray analysis or NMR data. Preferably, such 3D data is of sufficient spatial resolution to allow identification of the target residues.

If only a primary structure (sequence) of the target protein is available, technique such as homology modeling might be used. Accessible surface area in this case is determined using homology-based modeling from known 3D structures of proteins or their domains. Any suitable tool for prediction of 3D structure might be used. Such tools are well-known in the field. Examples of such tools are MOE, Schrodinger MAESTRO or Bioluminate, Modeller, i-TASSER, Rosetta, Phyre2. Such model in that case could be used to identify surface-accessible residues.

Hence, the present disclosure provides a method for identifying groups of amino-acid residues (patches) for substitution useful for determination of the importance of such residues for binding to a molecule of interest, said method comprising:
a) obtaining 3D structure information for the target protein;
b) identifying, using obtained 3D structural data, the amino-acid residues which are within the accessible surface area;
c) for each of the identified amino-acids selecting 1 or more amino-acids which are within a predetermined distance from the identified amino-acid and are within the accessible surface area, whereby such combination of amino-acid residues forms a patch of 2 or more amino acids (patch);
d) selecting, from the large number of generated possible patches, a set of representative patches that cover the majority of the target protein's accessible surface area, while minimizing the number of patches likely to cause the target protein to misfold.

In particular, such pre-determined distance is 4, 4.5, 5, 5.5, 6, 6.5, or 7 Å. Preferably, such distance is between 6 and 6.5 Å. Preferably, alanines and glycines are not selected for substitution. Depending on the relevance of Cys residues in the 3D structure such can be either substituted or not selected for substitution. Cys is often involved into formation of S—S bonds in proteins and is important for tertiary structure. Gly is a very flexible amino acid and substituting such with a larger amino acid such as Ala may also have a structural effect. Optionally, Pro residues can also be left out of the analysis as such are often involved in secondary structure formation.

More specifically the amino-acids within the accessible surface area in step (b) are selected based on the calculated solvent-accessible surface area of side chains. Standard methods to calculate solvent accessibility can be applied. In a typical example a probe of 1.4 Å is used for calculations (a simplified version of H₂O molecule wherein such probe has a size similar to an H₂O molecule). In such calculations atoms of the amino-acid residues that touch the probe are classified as surface accessible atoms. Surface accessibility of each amino-acid is calculated in A². Subsequently a ratio between the actual surface exposed area (in Å²) and theoretical probable surface exposure (in Å²) is calculated. Different cut-offs can be selected depending on the desired accuracy and the size of the protein. The larger the protein, the higher might be the cut-off selected. Such can be selected from 0.5 (50%), 0.2 (20%), preferably such cut-off is between 0.05(5%)-0.1(10%), more preferably such cut-off is 0.07 (7%). Such filtering step is useful to eliminate potentially misfolding proteins.

Further steps to reduce the amount of misfolded proteins in the final array are performed. For example, residues that cause breakage of more than one hydrogen bond between any of the original residues of each mutated patch (2 or more residues) and the rest of the protein are avoided. In another embodiment, residues that cause breakage of more than two hydrogen bonds within the protein are also avoided. Similarly, any breakage in the salt-bridges should also be preferably avoided. Additionally, mutations that expose large hydrophobic areas of the protein are also avoided.

Hence, in a preferred embodiment of the method, the method excludes or filters out 1) patches that result in the breakage of hydrogen bonds (preferably maximum of 2 broken bonds allowed) and 2) salt bridges (preferably maximum 1 broken bond allowed), as well as 3) the exposure of large hydrophobic patches (preferably maximum 15 Å² of exposed hydrophobic surface allowed).

Optionally, further granularity can be achieved by performing a molecular dynamics simulation with any widely used simulations package (e.g. AMBER, GROMACS, DESMOND, etc.) with a subsequent analysis of interaction persistence. Hydrogen bonds and salt bridges that are present in a large fraction of the simulation trajectory can be considered “essential” and should not be broken by an Ala mutation, whereas bonds that are only observed in a small fraction of the simulation are likely to have little impact on the protein's stability.

Additionally, after all the patches of residues have been identified any redundancy in such is eliminated by eliminating the patches that generate redundancy. This step is optional as it could be beneficial to have some redundancy in the coverage of the accessible surface area, however having such redundancy might provide technical difficulty in generating mutant clones subsequently. Hence, such redundancy should be considered in the context of the protein size, complexity and technical limitations in designing the corresponding mutant proteins.

Ideally, the steps above are performed for the whole protein surface to make sure that maximum surface-accessible area is covered by the identified patches. It would be preferable to avoid having some parts of the surface-accessible area not covered by such patches. The purpose is to cover the solvent accessible surface while minimizing the number of generated misfolded proteins.

If, for example, using patches of 2 substitutions would not cover the whole surface-accessible area, additional patches consisting of 3 substitutions can be designed. Larger patches of more than 3 substitutions can also be used, however going beyond 3 substitutions may lead to misfolding of the target protein, in other words and increased percentage of misfolded mutant proteins will be generated. Hence, preferably patches containing 2 or 3 Ala substitutions are used. If desired additional single Ala substitution could also be selected. However, such may not provide the desired sensitivity compared to 2 or more substitutions. In the case of single substitutions only steps (a) and (b) above are performed. Preferably such substitutions are Ala substitutions.

Finally, after identifying patches for substitution, a list of proteins with such patches containing such substitutions is produced. Typically such are output in a FASTA file format or a PYMOL session file.

### Arrays of Mutant Proteins

The generated sequences of mutated target protein of interest are subsequently produced for experimental testing. A typical way to produce such is by cloning the sequences into a suitable expression vector. As a control, the wild type sequence of the target protein of interest is also cloned.

An array of mutant proteins can be produced using techniques known to the skilled person. Any suitable expression system for expressing proteins in target cells can be used. Preferably a mammalian cell system is used for expressing cloned mutant peptides. Mammalian cells would allow for the mutant polypeptides to be secreted out of such cells and make testing such peptides easier. Any mammalian cell or cell line could be used as long as such allows for sufficient expression of each of the mutant peptides. In such a mammalian system a suitable expression vector can be used. Many mammalian expression vectors are commercially available. Typically such a vector will comprise a constitutive promoter, such as cytomegalovirus (CMV) promoter.

Alternatively, cell-free expression systems can be used. Such systems can useful for studying cytoplasmic protein-protein interactions. Cell free expression could be an ideal method for such proteins and does not require lysis of any cells.

Alternatively, cell-surface expression arrays can also be used. There are ways to estimate affinity on binding to target protein expressed on cells (e.g. a Ligand-Tracer device or by doing flow cytometry titrations). This would be useful for targets that are not easily expressed in solution, e.g. ion channels and G-protein receptors (GPCRs). This could also include the use of other sources of membrane preparations such as Virus-like particles (VLPs), amphipols or SMALPs which can also help maintain membrane protein fold.

Alternatively, a bacterial cell and a suitable vector for expression can be used. However, purifying such cloned mutant proteins from bacterial cells would be more complex.

When using a mammalian expression vector in mammalian cells each of the mutant proteins might be fused to a signal peptide for the export of such proteins out of such cells. More specifically a signal peptide comprising sequence MEWSWVFLFFLSVTTGVMA (SEQ ID NO: 1) can be used.

Additionally, if desired, such mutant proteins can be fused to a molecule or a protein that allows for easier binding of such fusion proteins to a carrier surface for further testing. Example of such a protein is biotin that can be easily captured by streptavidin. Mutant proteins can further be fused to such a protein using a linker sequence, such as for example a His tag or bacterial Avi tag. The choice of appropriate tag and linker sequence will depend on the mutant proteins as well as the choice of target cells used for expression.

In a particular embodiment, each of the mutant proteins are fused to an Fc region, preferably human Fc domain (SEQ ID NO: 2: APELLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTLPPS RDELTKNQVSLTCLVKGFYPSDIAVEWESNGQPENNYKTTPPVLDSDGSFFLYSKLTVDKSR WQQGNVFSCSVMHEALHNHYTQKSLSLSPGK). The Fc domain in such fusion protein is fused to the mutant protein using a Fc hinge region DKTHTCPPCP (SEQ ID NO: 3). Use of Fc domain in such fusion proteins offers practical advantages, such as higher robustness in detection and ease of capturing such fusion proteins on a surface. Optionally one or more linker sequences can be introduced into the fusion protein sequence between the Fc domain and the target mutant protein if necessary, such as triple Ala linker.

Preferably, such fusion proteins comprising human Fc domain are expressed in in mammalian Expi293 cells, or any other cells that can generate sufficient concentration of the protein.

Optionally, proteins that might potentially misfold could be removed from the array by pre-screening the array using polyclonal antibodies (targeting multiple epitopes) against the target protein or any commercial monoclonal antibodies of known epitopes which are suitable for ELISA assays (as such antibodies would recognize a structural epitope).

Hence, the present disclosure provides an array of proteins, optionally fused to another protein as described herein, wherein such array comprises a multiplicity of mutant protein sequences of a target protein, each such protein comprising a patch of 2 or more amino-acid substitutions for another amino-acid (patch) compared to the parent sequence of the target protein, and wherein such substitutions have been introduced into the residues of the accessible surface area, and wherein the residues in each patch are within a predetermined spatial distance from each other.

In yet another embodiment the array comprises a multiplicity of mutant protein sequences of a target protein, each such protein comprising a patch of 2 or 3 amino-acid substitutions for another amino-acid. Preferably each such protein comprises a patch of 2 amino-acid substitutions for another amino-acid. Alternatively each such protein comprises a patch of 3 amino-acid substitutions for another amino-acid. Preferably, such substitutions are Ala substitutions. Preferably, Cys, Ala and Gly residues in the parent sequence are not substituted. Preferably the residues are further selected based on the criteria as described above. Preferably the array comprises the wild type target protein as well as a control.

### Measuring Binding Properties of Mutant Proteins

Finally, binding properties of a molecule of interest to each of the mutant target proteins on the array are measured. Such measurements can be performed using any suitable method available. Preferably, such measurements are performed using a high-throughput method.

The affinity of a molecule of interest, as well as the extent to which such molecule inhibits binding to the target protein, can be determined by one of ordinary skill in the art using conventional techniques, for example those described by Scatchard et al. (Ann. KY. Acad. Sci. 51:660-672 (1949)) or by surface plasmon resonance (SPR) using systems such as BIAcore. For surface plasmon resonance, mutant proteins are immobilized on a solid phase and exposed to ligands and/or the molecule of interest in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

Alternative platforms using techniques similar to SPR are provides by Cartera (carterra-bio.com) such as Carterra LSA Platform. It is a high throughput antibody characterization platform that combines flow printing microfluidics with high throughput surface plasmon resonance (SPR) detection technology.

Other types of platforms include techniques utilizing cell surface-expression arrays. An example of such platform is Ligand Tracer (ligandtracer.com) which is particularly suited to follow protein binding to cell-surface receptors and allows to measure on- and off-rates as well as affinities.

In order to simplify the measurements, each of the mutant proteins of the array could be fused to a molecule or a protein to allow to capture such on a surface for easier detection of binding properties.

The molecule of interest can be any molecule of a size that allows for interaction with multiple (2 or more) residues on the surface of the target protein. Preferably such molecule of interest is a protein, more preferably such molecule of interest is an antibody. Alternatively, such molecule of interest if a ligand or a receptor of the target protein.

Antibodies include whole antibodies and functionally active fragments thereof (i.e., molecules that contain an antigen binding domain that specifically binds to the target protein, also termed antigen-binding fragments). Antibodies might be monoclonal antibodies.

Preferably the binding to each of the mutant proteins is determined using Bio-Layer Interferometry (BLI) is a label-free technology. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time (REF)

Typically arrays of 30, 60 cloned mutant proteins are used. However the size of such arrays depends on the size of the target protein and the desired coverage of the solvent-accessible area. Preferably the mutant proteins are provided on a 96 well plate or 384-well plate. Generally a BLI instrument can handle 96- or 384-well plates for measurements.

When using BLI technology typically each sensor is exposed to a solution containing the molecule of interest (such as an antibody or a ligand) for which the binding site is being determined. The advantage of BLI technology is that is almost as sensitive as a normal BIACore, it is high throughput (96 clones can be tested at the same time) and uses disposable sensor tips so there is no need to regenerate the surface and reuse a chip as you would typically do with BIACore.

Different measurements of binding of the molecule of interest to the mutant proteins can be used to determine which of the mutant proteins demonstrate reduced binding. Typically, dissociation constants or binding constants are measured. Typically complete loss of binding or how quickly the molecule of interest is coming off the mutant protein can be measured. Appropriate controls are generally used when measuring the binding properties of the molecule of interest. Commonly the binding properties are compared to parental sequence of the target protein (wild type, WT). Typically the majority of mutant proteins will show the same K_{d} as the WT. The mutant proteins showing a difference in binding should be considered. Typically, any dissociation constant difference of at least 2, 3, 4, 5, 6, 7, 8, 9, or 10 or more fold compared to wild-type target protein is considered. Preferably any difference of at least 3-fold is considered significant. The mutant proteins that produce the results with low noise to signal resolutions are ignored or re-measured.

If desired mutant proteins comprising patches of different size, such as patches of 2 or 3 substitutions can be used on an array. Mutant proteins comprising single substitutions can also additionally be tested for binding properties if a higher precision is required, provided such provide sufficient sensitivity to obtain a measurable effect.

### Implementation of the Methods

It will be appreciated that the disclosure also applies to computer programs, particularly computer programs on or in a carrier, adapted to put the methods of the present disclosure into practice. Specific computational or analytical steps of the method provided by the present disclosure can be implemented using a computer. Hence the present disclosure provides a computer-implemented method for identifying amino-acid residues for substitution useful for determination of the importance of such residues for binding to a molecule of interest, said method comprising:
a) receiving 3D structure information data for the target protein;
b) identifying, using obtained 3D structural data, the amino-acid residues which are within the accessible surface area;
c) for each of the identified amino-acids selecting 1 or more amino-acids which are within a predetermined distance from the identified amino-acid and are within the accessible surface area, whereby such combination of amino-acid residues forms a patch of 2 or more amino acids (patch);
d) selecting, from the large number of generated possible patches, a set of representative patches that cover the majority of the target protein's accessible surface area, and filtering out patches likely to cause the target protein to misfold.
e) outputting a list of mutated sequences based on the identified patches, each of such sequences containing one patch.

Different aspects of the steps of the method can be supplemented by additional sub-steps as described herein. Specific selection criteria can also be implemented in a computer program. Such criteria are described above in relation to the method for identifying groups of amino-acid residues (patches) for substitution useful for determination of the importance of such residues for binding to a molecule of interest. Such computer program could have an interface and provide both a visual output on a screen and an output in a form of a text file or a file of an appropriate format.

The present disclosure further provides a computer program comprising code means for performing the steps of the method described above, wherein said computer program execution is carried out on a computer. The present disclosure further provides a non-transitory computer-readable medium storing thereon executable instructions, that when executed by a computer, cause the computer to execute the method as described above.

The computer program may be in the form of a source code, an object code, a code intermediate source. The program can be in a partially compiled form, or in any other form suitable for use in the implementation of the method and its variations according to the present disclosure. Such program may have many different architectural designs. A program code implementing the functionality of the method according to the disclosure may be sub-divided into one or more sub-routines. Many different ways of distributing the functionality among these sub-routines exist and will be known to the skilled person. The sub-routines may be stored together in one executable file to form a self-contained program. Alternatively, one or more or all of the sub-routines may be stored in at least one external library file and linked with a main program either statically or dynamically, e.g. at run-time. The main program contains at least one call to at least one of the sub-routines. The sub-routines may also call each other.

The present disclosure further provides a computer program product comprising computer-executable instructions implementing the steps of the methods set forth herein or its variations as set forth herein. These instructions may be sub-divided into sub-routines and/or stored in one or more files that may be linked statically or dynamically. Another embodiment relating to a computer program product comprises computer-executable instructions corresponding to steps of the methods set forth herein. These instructions may be sub-divided into sub-routines and/or stored in one or more files.

### Examples

### Example 1. Identifying Target Residues for Substitution

Using a published structure of TREM1 (PDB code: 1SMO, chain A) the surface accessible residues were identified using the software PSA, which is part of the JOY software suite for protein structure annotation (https://doi.org/10.1093/bioinformatics/14.7.617). Residues were classified as accessible if their relative side chain accessibility was at least 7%. Cysteines, glycines and alanines are not considered. Each of the selected residues were considered in turn and the amino acids within the previously selected set that have a sidechain heavy atom within 6 Angstroms of the central residue's sidechain heavy atoms were selected. All possible combinations of 2 and 3 residues within this selected patch of residues is subsequently listed (all combinations had to include the central residue). Each of these potential combinations of residues are assessed for breakage of hydrogen bonds or salt bridges. Any such combinations of residues that broke a hydrogen bond or a salt bridge with a residue outside of these 2 or 3 residues was discarded (i.e. hydrogen bonds within the selected patch of 2 or 3 residues were not considered). For such purpose a hydrogen bond was simply defined here as any oxygen atom within 3.5 Angstroms of another oxygen or nitrogen atom. Each residue in the sequence was indexed as being hydrogen bonded if its sidechain formed a hydrogen bond, as defined above, with a sidechain or mainchain atom of another residue, but excluding the mainchain of the neighboring residues in the amino acid sequence.

A salt bridge was defined as a Lysine, Arginine or Histidine's sidechain nitrogen atom within 4 Angstroms of an Aspartate or Glutamate's sidechain oxygen atom.

Furthermore, any 2 or 3 alanine patches whose mutation resulted in an increase in the protein's hydrophobic surface of more than 15 Å² are discarded. The hydrophobic surface is calculated as the sum of all non-polar sidechain atoms' surface areas within the protein (the solvent-accessible non-polar sidechain surface for each amino acid is provided by the software PSA).

All remaining 2 Ala patches were taken forward. Only two 3 Ala patches and two 1 Ala mutants were taken forward, as they covered residues that had not made it into the final set of 2 Ala patches. The 3 Ala and 1 Ala patches overlapped (provided some redundancy) but were all taken forward, to balance the risk of misfolding and the risk of producing a weak effect on binding that would fall below the detection limit of the experiment.

All the selected patches of residues were computationally converted into alanines and the corresponding protein sequences written into a FASTA file. The corresponding 3D models were written into a PyMOL session file for visual inspection.

The protein sequences were then used for production of the corresponding DNA sequence for expression of such proteins. Table 3 provides the list of the protein sequences designed for the array.

### Example 2. Generation of an Array of Fusion Proteins

Once the array was designed then the mutant proteins were ordered as cloned a standard Fc mammalian vector (KAN resistance, CMV promoter and features included were standard) and ready for transfection and expression. In this example each of the mouse TREM1 proteins was fused to human Fc:

MEWSWVFLFFLSVTTGVMA AIVLEEERYDLVEGQTLTVKCPFNI MKYANSQKAWQRLPDGKEPLTLVVTQRPFTRPSEVHMGKFTLKHDPSEAMLQVQMT DLQVTDSGLYRCVIYHPPNDPVVLFHPVRLVVTKG AAA DKTHTCPPCP APELLGGPSVFLEPPKPKDTLIVIISRTPEVTCVVVDVSHEDPEVKFNWYVDGVEVHNAKTKPREEQ YNSTYRVVSVLTVLHQDWLNGKEYKCKVSNKALPAPIEKTISKAKGQPREPQVYTLPPSRDELTKNQ VSLTCLVKGFYPSDIAVEWESNGQPENNYKTTPPVLDSDGSFFLYSKLTVDKSRWQQGNVFSCSVM HEALHNHYTQKSLSLSPGK-(SEQ ID NO: 4). Underlined the signal peptide followed by the mouse TREM1 IgV domain (in bold), triple alanine linker (underlined), the hinge sequence (in grey) and in the human Fc (CH2CH3) sequence (in italic).

Lyophilized DNA was provided for each of the clones in a 96 well plate format which is resuspended in H₂O and 1 ug of that is used to transfect 1 ml of Expi293 mammalian cells. Following transfection, the cells were incubated at 37 C for 6 days (96 deep well format with 1 ml cell culture per well is used) to produce the protein Fc fused constructs in the supernatant. On day 6 the cells were spun down (at 4000 rpm), and the supernatant is transferred to a new tube (the remaining cell pellet in the original tube is discarded). Each culture supernatant represents an array clone and it is diluted in ¼ with PBST (0.05% Tween) buffer before capturing on the BLI sensors (coated with anti-human Fc antibody).

### Example 3. Measuring Binding Properties of the Fusion Proteins and Identifying the Epitope Residues

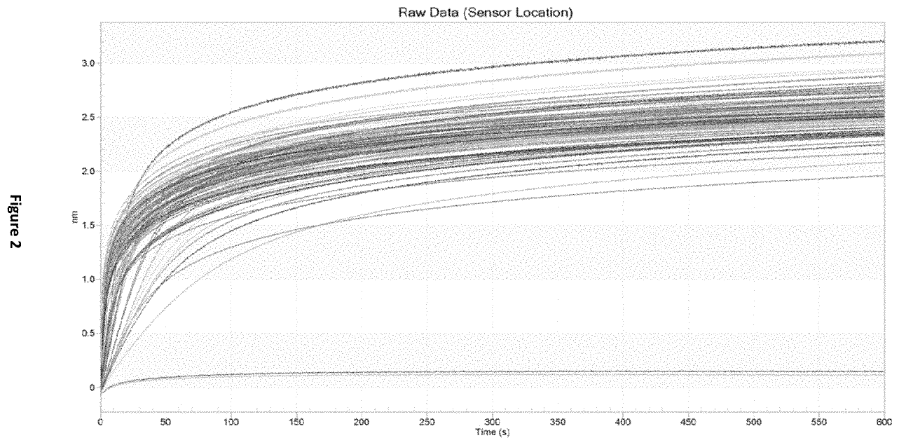

Capturing of each of the protein Fc constructs on the sensors tips can be monitored using BLI and a successful capturing is demonstrated by an increase in the nm signal. This is similar to Biacore and the change in the refractive index upon binding. In this case, capturing of the protein on the BLI sensor is accompanied by a shift in the interference pattern which can be measured in real time (as shown in FIG. 2). Capturing of 83 TREM-Fc protein clones on a sensor is accompanied by a signal increase which is measured in nm. For the 2 control samples there is no increase at all (mock).

Following capturing of the array clones and if it's the first time the array is used the clones are tested for binding to an existing polyclonal antibody and/or a monoclonal/s of known epitopes (known source of immunogen). An example of such antibody used in this example is the Monoclonal Anti-TREM1 antibody produced in mouse clone 2E2 (Sigma, WH0054210M4). The antibodies chosen must be suitable for ELISA and not for western blot only. Following such testing, any clones that fail to give signal for all the tested antibodies are excluded as being misfolded.

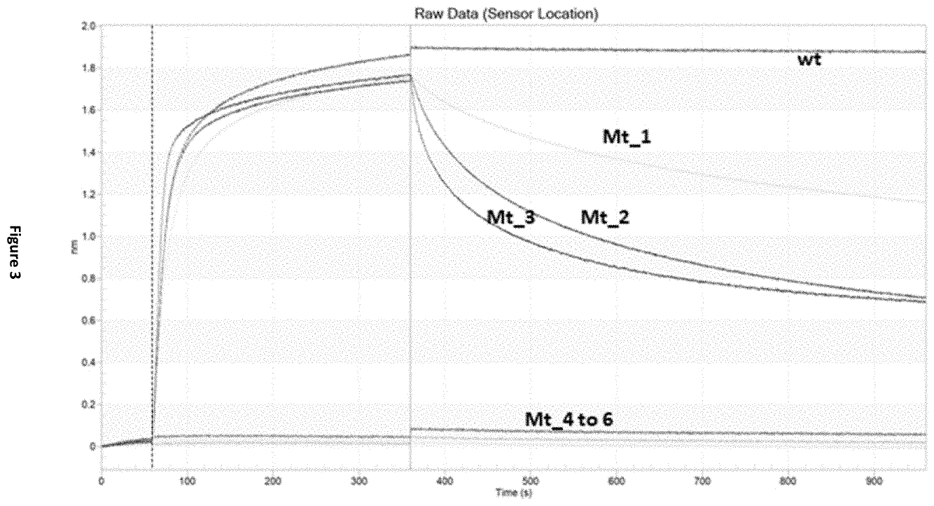

An example of an epitope mapping using this methodology is shown in FIG. 3:The kinetics for the wt clone is shown on the top and below that the dissociation constants of some mutant clones are shown. There is a clear difference in the dissociation constants compared to the wt indicating that these mutations represent epitope residues for that antibody. In this case some mutations cause a complete loss of binding (named mutants 4 to 6 for convenience). A 1:1 fitting model can be applied to the above and the values for each of the dissociation rates can be exported and compared in an excel table for confirmation.

After exclusion of misfolded proteins, the array is used in the same way with the antibody of interest for which the epitope is not known. The binding of the antibody for each of the array clones is monitored and clones that show no binding or binding with reduced dissociation rates are identified as containing epitope residues. The dissociation rate is measured in sec′ and the bigger that value is the faster the antibody is dissociating from its target (more info here: https://www.sprpages.nl/kinetics/dissociation). The dissociation constant of each mutant array clone is always comparted to that of the parental and the vast majority of them with the exception of the ones that contain epitope mutated residues should give dissociation rates similar to the parental one.

An example comparison of different Ala arrays for an exemplary molecule of interest is provided in Table 2 below.

TABLE 2

| | Number of | |
|---|---|---|
| | residues | |
| | identified as | |
| | target binding | |
| Array | region | Notes |
| 3 Ala array | 2 | Only 2 residues next to each other |
| | | in the sequence were identified as |
| | | the binding region |
| 2 Ala array | 3 | The residues were identified in |
| | | different locations of the |
| | | sequence; one of the residues is |
| | | the same as identified using 3 Ala |
| | | array |
| 1 Ala array | 0 | No epitope residues were |
| | | identified |

TABLE 3
List of TREM1 mutant proteins used to design the array

| WT | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
|---|---|
| 1_2 | AAAALTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
| 2_3 | AATAATEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
| 3_4 | AATKAAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
| 4_103 | AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHALFDRIRLVVT |
| 4_105 | AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLADRIRLVVT |
| 4_106 | AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFARIRLVVT |
| 6_8 | AATKLTEAKAELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
| 6_106 | AATKLTEAKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFARIRLVVT |
| 7_9 | AATKLTEEAYALKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
| 10_11 | AATKLTEEKYEAAEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
| 11_12 | AATKLTEEKYELAAGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
| 11_14 | AATKLTEEKYELAEGATLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVT |
| 12_84 | AATKLTEEKYELKAGQTLDVKCDYTLEKFASSQKAWQIIRDGEMPKTLACTERPSKNSHPVQVGRIILEDYHDHGLLRVRMVNLQAEDSGLYQCVIYQPPKEPHMLFDRIRLVVT |
| 15_80 | AATKLTEEKYELKEGQALDVKCDYTLEKFASSQKAWQIIRDGEMPKTLACTERPSKNSHPVQVGRIILEDYHDHGLLRVRMANLQVEDSGLYQCVIYQPPKEPHMLFDRIRLVVT |
| 19_74 | AATKLTEEKYELKEGQTLDVACDYTLEKFASSQKAWQIIRDGEMPKTLACTERPSKNSHPVQVGRIILEDYHDHGALRVRMVNLQVEDSGLYQCVIYQPPKEPHMLFDRIRLVVT |
| 23_24 | AATKLTEEKYELKEGQTLDVKCDYAAEKFASSQKAWQIIRDGEMPKTLACTERPSKNSHPVQVGRIILEDYHDHGLLRVRMVNLQVEDSGLYQCVIYQPPKEPHMLFDRIRLVVT |
| 23_25 | AATKLTEEKYELKEGQTLDVKCDYALAKFASSQKAWQIIRDGEMPKTLACTERPSKNSHPVQVGRIILEDYHDHGLLRVRMVNLQVEDSGLYQCVIYQPPKEPHMLFDRIRLVVT |
| 23_26 | AATKLTEEKYELKEGQTLDVKCDYALEAFASSQKAWQIIRDGEMPKTLACTERPSKNSHPVQVGRIILEDYHDHGLLRVRMVNLQVEDSGLYQCVIYQPPKEPHMLFDRIRLVVT |
| 24_25 | AATKLTEEKYELKEGQTLDVKCDYTAAKFASSQKAWQIIRDGEMPKTLACTERPSKNSHPVQVGRIILEDYHDHGLLRVRMVNLQVEDSGLYQCVIYQPPKEPHMLFDRIRLVVT |
| 25_26 | AATKLTEEKYELKEGQTLDVKCDYTLAAFASSQKAWQIIRDGEMPKTLACTERPSKNSHPVQVGRIILEDYHDHGLLRVRMVNLQVEDSGLYQCVIYQPPKEPHMLFDRIRLVVT |
| 29_30 | AATKLTEEKYELKEGQTLDVKCDYTLEKFAAAQKAWQIIRDGEMPKTLACTERPSKNSHPVQVGRIILEDYHDHGLLRVRMVNLQVEDSGLYQCVIYQPPKEPHMLFDRIRLVVT |
| 29_52 | AATKLTEEKYELKEGQTLDVKCDYTLEKFAASQKAWQIIRDGEMPKTLACTERASKNSHPVQVGRIILEDYHDHGLLRVRMVNLQVEDSGLYQCVIYQPPKEPHMLFDRIRLVVT |
| 36_37 | AATKLTEEKYELKEGQTLDVKCDYTLEKFASSQKAWQAARDGEMPKTLACTERPSKNSHPVQVGRIILEDYHDHGLLRVRMVNLQVEDSGLYQCVIYQPPKEPHMLFDRIRLVVT |
| 36_38 | AATKLTEEKYELKEGQTLDVKCDYTLEKFASSQKAWQAIADGEMPKTLACTERPSKNSHPVQVGRIILEDYHDHGLLRVRMVNLQVEDSGLYQCVIYQPPKEPHMLFDRIRLVVT |
| 36_44 | AATKLTEEKYELKEGQTLDVKCDYTLEKFASSQKAWQAIRDGEMPATLACTERPSKNSHPVQVGRIILEDYHDHGLLRVRMVNLQVEDSGLYQCVIYQPPKEPHMLFDRIRLVVT |
| 36_46 | AATKLTEEKYELKEGQTLDVKCDYTLEKFASSQKAWQAIRDGEMPKTAACTERPSKNSHPVQVGRIILEDYHDHGLLRVRMVNLQVEDSGLYQCVIYQPPKEPHMLFDRIRLVVT |
| 37_43 | AATKLTEEKYELKEGQTLDVKCDYTLEKFASSQKAWQIARDGEMAKTLACTERPSKNSHPVQVGRIILEDYHDHGLLRVRMVNLQVEDSGLYQCVIYQPPKEPHMLFDRIRLVVT |
| 37_89 | AATKLTEEKYELKEGQTLDVKCDYTLEKFASSQKAWQIARDGEMPKTLACTERPSKNSHPVQVGRIILEDYHDHGLLRVRMVNLQVEDSGAYQCVIYQPPKEPHMLFDRIRLVVT |
| 41_42 | AATKLTEEKYELKEGQTLDVKCDYTLEKFASSQKAWQIIRDGAAPKTLACTERPSKNSHPVQVGRIILEDYHDHGLLRVRMVNLQVEDSGLYQCVIYQPPKEPHMLFDRIRLVVT |
| 41_44 | AATKLTEEKYELKEGQTLDVKCDYTLEKFASSQKAWQIIRDGAMPATLACTERPSKNSHPVQVGRIILEDYHDHGLLRVRMVNLQVEDSGLYQCVIYQPPKEPHMLFDRIRLVVT |
| 42_43 | AATKLTEEKYELKEGQTLDVKCDYTLEKFASSQKAWQIIRDGEAAKTLACTERPSKNSHPVQVGRIILEDYHDHGLLRVRMVNLQVEDSGLYQCVIYQPPKEPHMLFDRIRLVVT |
| 42_100 | AATKLTEEKYELKEGQTLDVKCDYTLEKFASSQKAWQIIRDGEAPKTLACTERPSKNSHPVQVGRIILEDYHDHGLLRVRMVNLQVEDSGLYQCVIYQPPKAPHMLFDRIRLVVT |
| 43_44 | AATKLTEEKYELKEGQTLDVKCDYTLEKFASSQKAWQIIRDGEMAATLACTERPSKNSHPVQVGRIILEDYHDHGLLRVRMVNLQVEDSGLYQCVIYQPPKEPHMLFDRIRLVVT |
| 44_46 | AATKLTEEKYELKEGQTLDVKCDYTLEKFASSQKAWQIIRDGEMPATAACTERPSKNSHPVQVGRIILEDYHDHGLLRVRMVNLQVEDSGLYQCVIYQPPKEPHMLFDRIRLVVT |
| 46_64 | AATKLTEEKYELKEGQTLDVKCDYTLEKFASSQKAWQIIRDGEMPKTAACTERPSKNSHPVQVGRAILEDYHDHGLLRVRMVNLQVEDSGLYQCVIYQPPKEPHMLFDRIRLVVT |
| 50_54 | AATKLTEEKYELKEGQTLDVKCDYTLEKFASSQKAWQIIRDGEMPKTLACTARPSANSHPVQVGRIILEDYHDHGLLRVRMVNLQVEDSGLYQCVIYQPPKEPHMLFDRIRLVVT |
| 55_56 | AATKLTEEKYELKEGQTLDVKCDYTLEKFASSQKAWQIIRDGEMPKTLACTERPSKAAHPVQVGRIILEDYHDHGLLRVRMVNLQVEDSGLYQCVIYQPPKEPHMLFDRIRLVVT |
| 57_58 | AATKLTEEKYELKEGQTLDVKCDYTLEKFASSQKAWQIIRDGEMPKTLACTERPSKNSAAVQVGRIILEDYHDHGLLRVRMVNLQVEDSGLYQCVIYQPPKEPHMLFDRIRLVVT |
| 58_59 | AATKLTEEKYELKEGQTLDVKCDYTLEKFASSQKAWQIIRDGEMPKTLACTERPSKNSHAAQVGRIILEDYHDHGLLRVRMVNLQVEDSGLYQCVIYQPPKEPHMLFDRIRLVVT |
| 58_60 | AATKLTEEKYELKEGQTLDVKCDYTLEKFASSQKAWQIIRDGEMPKTLACTERPSKNSHAVAVGRIILEDYHDHGLLRVRMVNLQVEDSGLYQCVIYQPPKEPHMLFDRIRLVVT |
| 58_65 | AATKLTEEKYELKEGQTLDVKCDYTLEKFASSQKAWQIIRDGEMPKTLACTERPSKNSHAVQVGRIALEDYHDHGLLRVRMVNLQVEDSGLYQCVIYQPPKEPHMLFDRIRLVVT |
| 59_60 | AATKLTEEKYELKEGQTLDVKCDYTLEKFASSQKAWQIIRDGEMPKTLACTERPSKNSHPAAVGRIILEDYHDHGLLRVRMVNLQVEDSGLYQCVIYQPPKEPHMLFDRIRLVVT |
| 59_61 | AATKLTEEKYELKEGQTLDVKCDYTLEKFASSQKAWQIIRDGEMPKTLACTERPSKNSHPAQAGRIILEDYHDHGLLRVRMVNLQVEDSGLYQCVIYQPPKEPHMLFDRIRLVVT |
| 60_61 | AATKLTEEKYELKEGQTLDVKCDYTLEKFASSQKAWQIIRDGEMPKTLACTERPSKNSHPVAAGRIILEDYHDHGLLRVRMVNLQVEDSGLYQCVIYQPPKEPHMLFDRIRLVVT |
| 60_65 | AATKLTEEKYELKEGQTLDVKCDYTLEKFASSQKAWQIIRDGEMPKTLACTERPSKNSHPVAVGRIALEDYHDHGLLRVRMVNLQVEDSGLYQCVIYQPPKEPHMLFDRIRLVVT |
| 61_64 | AATKLTEEKYELKEGQTLDVKCDYTLEKFASSQKAWQIIRDGEMPKTLACTERPSKNSHPVQAGRAILEDYHDHGLLRVRMVNLQVEDSGLYQCVIYQPPKEPHMLFDRIRLVVT |
| 64_65 | AATKLTEEKYELKEGQTLDVKCDYTLEKFASSQKAWQIIRDGEMPKTLACTERPSKNSHPVQVGRAALEDYHDHGLLRVRMVNLQVEDSGLYQCVIYQPPKEPHMLFDRIRLVVT |
| 65_80 | AATKLTEEKYELKEGQTLDVKCDYTLEKFASSQKAWQIIRDGEMPKTLACTERPSKNSHPVQVGRIALEDYHDHGLLRVRMANLQVEDSGLYQCVIYQPPKEPHMLFDRIRLVVT |
| 71_72 | AATKLTEEKYELKEGQTLDVKCDYTLEKFASSQKAWQIIRDGEMPKTLACTERPSKNSHPVQVGRIILEDYHAAGLLRVRMVNLQVEDSGLYQCVIYQPPKEPHMLFDRIRLVVT |
| 72_74 | AATKLTEEKYELKEGQTLDVKCDYTLEKFASSQKAWQIIRDGEMPKTLACTERPSKNSHPVQVGRIILEDYHDAGALRVRMVNLQVEDSGLYQCVIYQPPKEPHMLFDRIRLVVT |
| 80_81 | AATKLTEEKYELKEGQTLDVKCDYTLEKFASSQKAWQIIRDGEMPKTLACTERPSKNSHPVQVGRIILEDYHDHGLLRVRMAALQVEDSGLYQCVIYQPPKEPHMLFDRIRLVVT |
| 84_85 | AATKLTEEKYELKEGQTLDVKCDYTLEKFASSQKAWQIIRDGEMPKTLACTERPSKNSHPVQVGRIILEDYHDHGLLRVRMVNLQAADSGLYQCVIYQPPKEPHMLFDRIRLVVT |
| 97_98 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQAAKEPHMLFDRIRLVVT |
| 98_99 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPAAEPHMLFDRIRLVVT |
| 99_100 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPAAPHMLFDRIRLVVT |
| 100_101 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKAAHMLFDRIRLVVT |
| 101_102 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEAAMLFDRIRLVVT |
| 102_103 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPAALFDRIRLVVT |
| 103_105 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHALADRIRLVVT |
| 105_106 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLAARIRLVVT |
| 7_9_111 | AATKLTEEAYALKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLAVTKG |
| 36_38_39 | AATKLTEEKYELKEGQTLDVKCDYTLEKFASSQKAWQAIAAGEMPKTLACTERPSKNSHPVQVGRIILEDYHDHGLLRVRMVNLQVEDSGLYQCVIYQPPKEPHMLFDRIRLVVTKG |
| 39 | AATKLTEEKYELKEGQTLDVKCDYTLEKFASSQKAWQIIRAGEMPKTLACTERPSKNSHPVQVGRIILEDYHDHGLLRVRMVNLQVEDSGLYQCVIYQPPKEPHMLFDRIRLVVTKG |
| 111 | AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLAVTKG |

### Example 4. Predicting Mutant Protein Misfolding

A patch array, containing only 3-Ala patches, was generated for the human protein TREM1 (based on the PDB structure 1Q8M, chain A) with an simpler version of the method described herein. The simple example the algorithm does not give any consideration to whether the introduction of a patch of alanine mutations results in the breakage of hydrogen bonds or salt bridges, or the exposure of large hydrophobic surface patches. Table 4 demonstrates that the methodology that takes those elements into consideration significantly reduces the number of misfolded mutant proteins when compared to the simple algorithm.

The simple algorithm used for comparison considers every surface residue with >10% sidechain surface exposure, as the “central” residue of a single 3-Ala patch. For each such “central” residue, all residues within a heavy-atom distance of 6 Å that have a sidechain exposure >30% are considered as potential partners to form a 3-Ala patch. These potential partner residues are sorted by their Alpha Carbon-Alpha Carbon distance to the central residue and the two residues with the smallest distances are chosen to form the final 3-Ala patch. Duplicate mutant sequences are eliminated.

Each mutant protein was made and tested for misfolding using polyclonal antibodies (targeting multiple epitopes) and/or multiple monoclonal antibodies against the target protein. As shown in Table 4, out of 84 proteins in the array, designed using the simple algorithm, 26 misfolded (˜31%).

A second set of sequences, containing only 3-Ala patches, was computed using the preferred algorithm provided by the present disclosure, which avoids 1) patches that result in the breakage of hydrogen bonds (max 2 broken bonds allowed) and 2) salt bridges (max 1 broken bond allowed), as well as 3) the exposure of large hydrophobic patches (max 15 Å² of exposed hydrophobic surface allowed). The distance threshold to define a patch was set to 6 Å and the minimal sidechain surface exposure was set to 7%. Two versions of this sequence dataset were created: one with the redundancy filter enabled (70 sequences); the other version of the sequence set was made with the redundancy filter disabled and included all possible surface patches that met the above criteria (174 sequences). The two sequence datasets, created with the full algorithm described in this document, eliminated the majority of the sequences that were known to misfold, reducing the expected misfolding rate from ˜31% to between 3 and 4%.

An additional dataset for another target, “protein X”, with much longer amino-acid sequence (−3 times), was produced using the preferred algorithm of the present disclosure that takes into account the breakage of hydrogen bonds or salt bridges, or the exposure of large hydrophobic surface patches. The parameters chosen were: max 2 hydrogen bonds broken, max 1 salt bridge broken, max 15 Å² exposure of hydrophobic patches allowed; the distance threshold was set to 6.5 Å and the minimal sidechain surface exposure was set to 20%. This resulted in an array of 96 mutant protein sequences (93 3-Ala patches and two 2-Ala patches, one wild-type), and zero misfolded proteins.

TABLE 4
Numbers of experimentally-identified
misfolded proteins in sequence datasets.

| | Total | Number of |
|---|---|---|
| | number | experimentally |
| | of proteins | identified misfolded |
| Sequence dataset | on the array | proteins |
| TREM1 3-ala array designed | 84 | 26 |
| with the “simple algorithm” | | |
| TREM1 3-ala sequences | 70 | 3* |
| designed with the algorithm | | |
| of the present disclosure; | | |
| redundancy filter enabled | | |
| TREM1 3-ala sequences | 174 | 6* |
| designed with the algorithm | | |
| of the present disclosure; | | |
| redundancy filter disabled | | |
| Protein X 3-ala array | 96 | 0 |
| designed with the algorithm | | |
| of the present disclosure | | |

*sequence sets that were never expressed; the corresponding number of misfolded proteins is estimate based on the overlap between the sequence dataset in question and the set of sequences known to misfold (the 26 misfolded mutant proteins designed with the “simple algorithm”).

1-16. (canceled)

17: A method of identifying amino-acid residues on a target protein that form a binding site of a molecule of interest, said method comprising:
a) obtaining 3D structure information for the target protein;
b) identifying, using obtained 3D structural data, the amino-acid residues which are within an accessible surface area;
c) for each of the identified amino-acid residues selecting 1 or 2 amino-acid residues which are within a predetermined distance from the identified amino-acid residue and are within the accessible surface area, whereby such a combination of the amino-acid residues forms a patch of 2 or 3 amino acids correspondingly;
d) selecting, from a large number of generated possible patches, a set of representative patches that cover a majority of the accessible surface area of the target protein, while minimizing a number of patches likely to cause the target protein to misfold by eliminating patches that result in:
i: a breakage of 3 or more hydrogen bonds in the target protein;
ii: a breakage of 2 or more salt bridges in the target protein; and
iii: an exposure of hydrophobic surface of the target protein above a predetermined threshold.
e) producing a set of mutant proteins, wherein each of the mutant proteins comprises a mutated sequence of the target protein, wherein each of the mutated sequences comprises a single mutated patch of amino acids identified in step (d), and wherein each of the amino acids of the patch is substituted by another amino-acid;
f) measuring binding properties of each of the mutant proteins; and
g) identifying the patches that demonstrate decreased binding properties of the molecule of interest to corresponding mutant protein comprising such patch, wherein the amino-acid residues in such patches are identified as being a part of a binding site of the molecule of interest.

18: The method of claim 17, wherein the decrease in binding properties in comparison to a wild type target protein is observed.

19: The method of claim 17, wherein the pre-determined distance is a distance between a sidechain heavy atom of the selected amino-acid reside and sidechain heavy atoms of the identified residue amino-acid.

20: The method of claim 19, wherein the distance is between 6 and 6.5 Å

21: The method of claim 17, wherein each of the amino acids of the patch is substituted by Ala.

22: The method of claim 17, wherein the predetermined threshold for the exposure of the hydrophobic surface is 15 Å².

23: The method of claim 17, wherein each of the mutant proteins in the produced set is fused to another protein.

24: The method of claim 23, wherein the other protein is a human Fc domain.

25: The method of claim 17, wherein the binding properties are measured using Bio-Layer Interferometry (BLI).

26: An array of proteins comprising:
a multiplicity of mutant protein sequences of a target protein, each target protein comprising a patch of 2 or 3 amino-acid substitutions for another amino-acid compared to a parent sequence of the target protein, and wherein the amino-acid substitutions have been introduced into amino-acid residues of an accessible surface area, and wherein the amino-acid residues in each patch are within a predetermined spatial distance from each other, and wherein the patches do not result in:
i: a breakage of 3 or more hydrogen bonds in the target protein;
ii: a breakage of 2 or more salt bridges in the target protein; and
iii: an exposure of hydrophobic surface of the target protein above a predetermined threshold.

27: The array of claim 26, wherein the pre-determined distance is a distance between a sidechain heavy atom of the selected amino-acid reside and sidechain heavy atoms of the identified residue amino-acid, and wherein the pre-determined distance is between 6 and 6.5 Å.

28: The array of claim 26, wherein at least one of the substitutions is for Ala.

29: The array of claim 26, wherein the predetermined threshold for the exposure of the hydrophobic surface is 15 Å².

30: The array of claim 26, wherein each of the mutant proteins is fused to another protein.

31: The array of claim 30, wherein the other protein is a human Fc domain.

32: The array of claim 26, wherein the target protein is TREM1.
